(19) United States
(12) Patent Application Publication
(10) Pub. No.: US 2021/0137126 A1
(43) Pub. Date: May 13, 2021

(54) USE OF PEPTIDES AS INSECTICIDAL AGENTS

(71) Applicants: INSTITUT NATIONAL DES SCIENCES APPLIQUEES DE LYON, VILLEURBANNE (FR); Institut National de Recherche Pour L'Agriculture, L'Alimentation et L'Environnement, PARIS (FR)

(72) Inventor: PEDRO DA SILVA, LYON (FR)

(21) Appl. No.: 17/258,175

(22) PCT Filed: Jul. 5, 2019
(86) PCT No.: PCT/FR2019/051679

§ 371 Date: Jan. 6, 2021

(30) Foreign Application Priority Data
Jul. 6, 2018 (FR) 1856262

(51) Int. Cl. A01N 63/50; C12N 15/82

(52) U.S. Cl. CPC A01N 63/50; C12N 15/8281; C12N 15/8286

(57) ABSTRACT

The present invention concerns insecticidal peptides and their uses, in particular as plant protection agents. It also concerns transgenic organisms, in particular transgenic plants, expressing said peptides.

The present invention relates to insecticidal peptides and their uses, in particular as plant protection agents.

### TECHNICAL PRIOR ART OF THE INVENTION

The economic impact of losses from harvests due to insect pests is extremely important and costs billions of dollars every year.

Currently, the protection of crops is almost exclusively based on chemical treatments, which are being used to a greater and greater extent, but are being widely criticized because of their persistence in the environment and their toxicity as regards non-target organisms such as amphibians, aquatic fauna, other insects, or in fact the agriculturalists using such products. Thus, the insecticides that are currently used the most against aphids, namely neonicotinoids, will very shortly be prohibited in the European Union because they have been shown to be hazardous to pollinators.

In addition to these social and health concerns, it should also be noted that the last few decades have seen the emergence of destructive insects that are capable of resisting conventional treatments.

The development of novel, effective insecticidal molecules of natural origin that are not toxic to human beings and which are more environmentally friendly appears to be indispensable in order to limit the use of conventional insecticides obtained from chemical synthesis.

### SUMMARY OF THE INVENTION

The aim of the present invention is to provide novel insecticidal agents which are effective, in particular against aphids, which can be used as plant protection agents and which are therefore suitable for replacing the current treatments using neonicotinoids.

Thus, the present invention concerns the use, as a pesticidal agent, preferably as an insecticidal agent, of a peptide comprising the sequence with formula (I)

[chemical structure]

in which
X₁ represents a sequence of 1 to 9 amino acids,
X₂ represents a sequence of 2 amino acids,
X₃ represents a sequence of 3 amino acids,
X₄ represents a sequence of 3 amino acids,
X₅ represents a sequence of 3 to 5 amino acids,
X₆ represents a sequence of 4 and 6 amino acids,
X₇ represents one amino acid,
X₈ represents a sequence of 9 amino acids,
X₉ represents a sequence of 2 amino acids,
X₁₀ represents a sequence of 2 to 16 amino acids,
or a salt of said peptide which is acceptable in terms of plant protection.

### Preferably,

X₁ represents the sequence (Z)ₘ-[G/S], preferably the sequence (Z)ₘ-G, where Z is an amino acid that is selected independently for each of the m occurrences and m is 0 or a whole number between 1 and 8; and/or

X₂ represents the sequence [F/Y]-Z, preferably the sequence F-Z, where Z is an amino acid; and/or

X₄ represents the sequence [S/D/N]-Z-Z, preferably [S/D]-Z-Z, and more particularly preferably the sequence S-Z-Z, where Z is an amino acid that is selected independently for each occurrence; and/or

X₅ represents the sequence (Z)ₙ-[L/V], preferably the sequence (Z)ₙ-V, where Z is an amino acid that is selected independently for each of the n occurrences and n is a whole number between 2 and 4, and/or X₆ represents the sequence [G/N]-(Z)ₚ, preferably N-(Z)ₚ, where Z is an amino acid that is selected independently for each of the p occurrences and p is a whole number between 3 and 5; and/or

X₈ represents the sequence Z-[E/Q]-Z-Z-Z-Z-Z-[D/G]-Z (SEQ ID NO: 7), preferably the sequence Z-E-Z-Z-Z-Z-Z-D-Z (SEQ ID NO: 8), where Z is an amino acid that is selected independently for each occurrence; and/or

X₉ represents the sequence [P/A]-[Y/H], preferably the sequence P-Y.

In particular

X₁ may represent the sequence Z₁-Z₂-Z₃-Z₄-Z₅-Z₆-Z₇-Z₈-Z₉, where
Z₁ is absent or is D, and is preferably D,
Z₂ is absent or is F or I, and is preferably F,
Z₃ is absent or is D, and is preferably D,
Z₄ is absent or is P or Y, and is preferably P,
Z₅ is absent or is T, N or H, and is preferably T,
Z₆ is absent or is E, T or Y, and is preferably E,
Z₇ is absent or is F, L or I, and is preferably F,
Z₈ is absent or is K, R or E, and is preferably K, and
Z₉ is G or S, preferably G; and/or

X₂ may represent the sequence [F/Y]-[P/E/Q/L], preferably F-P; and/or

X₃ may represent the sequence [I/K/R]-[E/Y/F]-[I/N/G/L], preferably I-E-I; and/or

X₄ may represent the sequence [S/D/N]-[K/T/N/R]-[Y/H/V/I], preferably S-K-Y; and/or

X₅ may represent the sequence Z₁₀-Z₁₁-Z₁₂-Z₁₃-Z₁₄, where
Z₁₀ is absent or is E, and is preferably absent,
Z₁₁ is absent or is Y, and is preferably absent,
Z₁₂ is A, N, Y or H, and is preferably A,
Z₁₃ is V or K, preferably V,
Z₁₄ is V or L, preferably V; and/or

X₆ may represent the sequence Z₁₅-Z₁₆-Z₁₇-Z₁₈-Z₁₉-Z₂₀ where
Z₁₅ is N or G, preferably N,
Z₁₆ is absent or is Y, and is preferably Y,
Z₁₇ is absent or is T, A, K or D, preferably T,
Z₁₈ is S, I, R or E, and is preferably S,
Z₁₉ is R, L, D, V, A or F, preferably R, and
Z₂₀ is P, H, A or V, preferably P; and/or

X₇ may represent Y, I, S or A, preferably Y.

X₈ may represent the sequence [V/I/S]-[E/Q]-[A/Y/D]-[A/H/D/R/K]-[K/S/A/R]-[E/M/Q/L]-[R/E/L/N/K]-[D/G]-[Q/L/H/M] (SEQ ID NO: 9), preferably V-E-A-A-K-E-R-D-Q (SEQ ID NO: 10); and/or

X₉ may represent the sequence [P/A]-[Y/H], preferably the sequence P-Y; and/or

X₁₀ may represent the sequence Z₂₁-Z₂₂-Z₂₃-Z₂₄-Z₂₅-Z₂₆-Z₂₇-Z₂₈-Z₂₉-Z₃₀-Z₃₁-Z₃₂-Z₃₃-Z₃₄-Z₃₅-Z₃₆, where
Z₂₁ is Y, R, A, Q or P, preferably Y,
Z₂₂ is D, T, E or N, preferably D,
Z₂₃ is absent or is G, and is preferably absent,
Z₂₄ is absent or is G, S, H, Q or K, and is preferably absent,
Z₂₅ is absent or is P or A, and is preferably absent,
Z₂₆ is absent or is A, E or S, and is preferably absent,
Z₂₇ is absent or is M, Q, E or V, and is preferably absent,
Z₂₈ is absent or is L, M, E or V, and is preferably absent,
Z₂₉ is absent or is M or L, and is preferably absent,
Z₃₀ is absent or is H, and is preferably absent,
Z₃₁ is absent or is N, Q or D, and is preferably absent,
Z₃₂ is absent or is F, and is preferably absent,
Z₃₃ is absent or is L, and is preferably absent,
Z₃₄ is absent or is T or S, and is preferably absent,
Z₃₅ is absent or is S or N, and is preferably absent,
Z₃₆ is absent or is P, and is preferably absent.

More preferably, the peptide is selected from the group constituted by
a peptide comprising, or consisting of, a sequence of amino acids selected from the group constituted by the sequences SEQ ID NO: 1 to 6, and
a peptide comprising, or consisting of, a sequence with formula (I) having at least 50%, preferably at least 80%, at least 85%, at least 90%, at least 95%, at least 98% or at least 99% sequence identity with one of sequences SEQ ID NO: 1 to 6, and having a pesticidal activity, preferably an insecticidal activity.

More particularly preferably, the peptide comprises, or consists of, a sequence of amino acids selected from the group constituted by the sequences SEQ ID NO: 1 to 6, preferably comprises, or consists of, a sequence of amino acids selected from the group constituted by the sequences SEQ ID NO: 1 to 4, and more particularly preferably comprises, or consists of, the sequence of amino acids SEQ ID NO: 1.

The peptide preferably has a size in the range 40 to 100 amino acids, preferably in the range 40 to 70 amino acids.

The peptide may be used as an insecticidal agent against hemiptera, lepidoptera, coleoptera and/or diptera, preferably against hemiptera and more particularly aphids, in particular Acyrthosiphon pisum.

The present invention also concerns a non-human host cell comprising a heterologous nucleic acid coding for a peptide as used in accordance with the invention, said nucleic acid being placed under the control of a transcriptional promoter enabling the expression of said nucleic acid in said cell.

The cell may be a plant cell, preferably a leguminous plant or cereal plant cell, and more particularly preferably a leguminous plant cell selected from the group constituted by soya, bean, pea, chickpea, peanut, lentil, alfalfa, feva bean and carob or a cereal plant cell selected from the group constituted by wheat and corn.

The cell may also be an entomopathogenic microorganism, preferably an entomopathogenic bacterium or fungus.

The present invention also concerns an entomopathogenic virus comprising a heterologous nucleic acid coding for a peptide as used in accordance with the invention, said nucleic acid being placed under the control of a transcriptional promoter enabling the expression of said nucleic acid in the insect infected by the virus.

The present invention also concerns a transgenic plant or multicellular plant structure comprising at least one cell in accordance with the invention, as well as a method for the production of such a transgenic plant, comprising introducing a heterologous nucleic acid coding for a peptide as used in accordance with the invention into a plant cell, and reconstituting said organism from said cell.

The present invention furthermore concerns a plant protection composition comprising at least one peptide as used in accordance with the invention or one of its acceptable salts in terms of plant protection, a host cell and/or a virus in accordance with the invention, and a support and/or excipient which is acceptable in terms of plant protection. The composition may furthermore comprise one or more additional plant protection agents, preferably selected from insecticides, bactericides, fungals, virucides, growth regulators or stimulators of the natural defenses of the plant.

### BRIEF DESCRIPTION OF THE DRAWINGS

FIG. 1: Sequence for BCR4 peptide showing the C1-05, C2-C4 and C3-C6 disulfide bridges.

FIG. 2: ESI-HRMS spectrum (A) and HPLC chromatogram (B) of BCR4 peptide (retention time 22.5 min).

FIG. 3: Multiple alignment for members of the BCR1-2-4-5 family

### DETAILED DESCRIPTION OF THE INVENTION

The inventors have identified a novel family of natural insecticidal peptides that may be used as an alternative to conventional chemical pesticides.

These peptides belong to a novel class of cysteine-rich proteins known as “Bacteriocyte Cysteine Rich” (BCRs) and recently identified in the pea aphid Acyrthosiphon pisum (Shigenobu et al. 2013). In this organism, there are seven BCRs containing 67 to 108 residues, with six or eight cysteine residues. They are coded by orphelin genes and exclusively expressed in the bacteriocytes of the aphids. These host cells harbor Buchnera aphidicola, the primary symbiotic bacterium of aphids. The BCR sequences do not exhibit any significant similarity with other sequenced proteins of the organisms and a comparative analysis has confirmed that these peptides are limited to the aphid line.

In the present application, the inventors have demonstrated that the BCR4 peptide, belonging to the BCR1-2-4-5 sub-family, has an exceptional insecticidal activity against the pea aphid with a range of activity (5-80 μM) similar to that of the AG41 peptide, a promising entomotoxic peptide promoter of plant origin (WO 2015/087238 A1). The peptides of this family thus constitute an effective alternative to the use of pesticides obtained by chemical synthesis.

In accordance with a first aspect, the present invention concerns the use, as a pesticidal agent, and more particularly as an insecticidal agent, of a peptide comprising, or consisting of, the sequence with formula (I)

[chemical structure]

in which

X₁ represents a sequence of 1 to 9 amino acids,

X₂ represents a sequence of 2 amino acids,

X₃ represents a sequence of 3 amino acids,

X₄ represents a sequence of 3 amino acids,

X₅ represents a sequence of 3 to 5 amino acids,

X₆ represents a sequence of 4 to 6 amino acids,

X₇ represents one amino acid,

X₈ represents a sequence of 9 amino acids,

X₉ represents a sequence of 2 amino acids,

X₁₀ represents a sequence of 2 to 16 amino acids,

or a salt of said peptide which is acceptable in terms of plant protection.

In the formula (I), the lines connecting the various cysteine residues represent the disulfide bridges.

In the present document, the terms “peptide”, “oligopeptide”, “polypeptide” and “protein” are used interchangeably and refer to a chain of amino acids linked via peptide linkages, irrespective of the number of amino acid residues constituting this chain.

As used here, the term “amino acid” refers to the 20 standard natural amino acid residues (G, P, A, V, L, I, M, C, F, Y, W, H, K, R, Q, N, E, D, S and T), to the rare natural amino acid residues (for example hydroxyproline, hydroxylysine, allohydroxylysine, 6-N-methyllysine, N-ethylglycine, N-methylglycine, N-ethyl asparagine, allo-isoleucine, N-methyl isoleucine, N-methylvaline, pyroglutamine or aminobutyric acid) and to non-natural amino acids (for example norleucine, norvaline and cyclohexylalanine). Preferably, this term refers to the standard 20 natural amino acid residues (G, P, A, V, L, I, M, C, F, Y, W, H, K, R, Q, N, E, D, S and T).

The amino acids are represented here by their single-letter or three-letter code in accordance with the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

The amino acids constituting the peptide used in accordance with the invention may have the L or D configuration, preferably the L configuration.

In accordance with one embodiment,

X₁ represents the sequence (Z)ₘ-[G/S], preferably the sequence (Z)ₘ-G, where Z is an amino acid that is selected independently for each of the m occurrences and m is 0 or a whole number between 1 and 8; and/or

X₂ represents the sequence [F/Y]-Z, preferably the sequence F-Z, where Z is an amino acid; and/or

X₄ represents the sequence [S/D/N]-Z-Z, preferably [S/D]-Z-Z, and more particularly preferably the sequence S-Z-Z, where Z is an amino acid that is selected independently for each occurrence; and/or

X₅ represents the sequence (Z)ₙ-[L/V], preferably the sequence (Z)ₙ-V, where Z is an amino acid that is selected independently for each of the n occurrences and n is a whole number between 2 and 4, and/or

X₆ represents the sequence [G/N]-(Z)ₚ, preferably N-(Z)ₚ, where Z is an amino acid that is selected independently for each of the p occurrences and p is a whole number between 3 and 5; and/or

X₈ represents the sequence Z-[E/Q]-Z-Z-Z-Z-Z-[D/G]-Z (SEQ ID NO: 7), preferably the sequence Z-E-Z-Z-Z-Z-Z-D-Z (SEQ ID NO: 8), where Z is an amino acid that is selected independently for each occurrence; and/or

X₉ represents the sequence [P/A]-[Y/H], preferably the sequence P-Y.

In accordance with one particular embodiment, X₁ represents the sequence Z₁-Z₂-Z₃-Z₄-Z₅-Z₆-Z₇-Z₈-Z₉, where
Z₁ is absent or is D, preferably is D,
Z₂ is absent or is F or I, preferably is F,
Z₃ is absent or is D, preferably is D,
Z₄ is absent or is P or Y, preferably is P,
Z₅ is absent or is T, N or H, preferably is T,
Z₆ is absent or is E, T or Y, preferably is E,
Z₇ is absent or is F, L or I, preferably is F,
Z₈ is absent or is K, R or E, preferably is K, and
Z₉ is G or S, preferably G; and/or

X₂ represents the sequence [F/Y]-[P/E/Q/L], preferably F-P; and/or

X₃ represents the sequence [I/K/R]-[E/Y/F]-[I/N/G/L], preferably I-E-I; and/or

X₄ represents the sequence [S/D/N]-[K/T/N/R]-[Y/H/V/I], preferably S-K-Y; and/or

X₅ represents the sequence Z₁₀-Z₁₁-Z₁₂-Z₁₃-Z₁₄, where
Z₁₀ is absent or is E, preferably is absent,
Z₁₁ is absent or is Y, preferably is absent,
Z₁₂ is A, N, Y or H, preferably is A,
Z₁₃ is V or K, preferably V,
Z₁₄ is V or L, preferably V; and/or

X₆ represents the sequence Z₁₅-Z₁₆-Z₁₇-Z₁₈-Z₁₉-Z₂₀, where
Z₁₅ is N or G, preferably N,
Z₁₆ is absent or is Y, preferably is Y,
Z₁₇ is absent or is T, A, K or D, preferably T,
Z₁₈ is S, I, R or E, and is preferably S,
Z₁₉ is R, L, D, V, A or F, preferably R, and
Z₂₀ is P, H, A or V, preferably P; and/or

X₇ represents Y, I, S or A, preferably Y; and/or

X₈ represents the sequence [V/I/S]-[E/Q]-[A/Y/D]-[A/H/D/R/K]-[K/S/A/R]-[E/M/Q/L]-[R/E/L/N/K]-[D/G]-[Q/L/H/M] (SEQ ID NO: 9), preferably V-E-A-A-K-E-R-D-Q (SEQ ID NO: 10); and/or

X₉ represents the sequence [P/A]-[Y/H], preferably the sequence P-Y; and/or

X₁₀ represents the sequence Z₂₁-Z₂₂-Z₂₃-Z₂₄-Z₂₅-Z₂₆-Z₂₇-Z₂₈-Z₂₉-Z₃₀-Z₃₁-Z₃₂-Z₃₃-Z₃₄-Z₃₅-Z₃₆, where
Z₂₁ is Y, R, A, Q or P, preferably Y,
Z₂₂ is D, T, E or N, preferably D,
Z₂₃ is absent or is G, preferably is absent,
Z₂₄ is absent or is G, S, H, Q or K, preferably is absent,
Z₂₅ is absent or is P or A, preferably is absent,
Z₂₆ is absent or is A, E or S, preferably is absent,
Z₂₇ is absent or is M, Q, E or V, preferably is absent,
Z₂₈ is absent or is L, M, E or V, preferably is absent,
Z₂₉ is absent or is M or L, preferably is absent,
Z₃₀ is absent or is H, preferably is absent,
Z₃₁ is absent or is N, Q or D, preferably is absent,
Z₃₂ is absent or is F, preferably is absent,
Z₃₃ is absent or is L, preferably is absent,
Z₃₄ is absent or is T or S, preferably is absent,
Z₃₅ is absent or is S or N, preferably is absent,
Z₃₆ is absent or is P, preferably is absent.

In accordance with another particular embodiment,

X₁ is selected from the group constituted by the sequences G, S, YHYIES (SEQ ID NO: 11), YFES (SEQ ID NO: 12), DFDPTEFKG (SEQ ID NO: 13), DIDPNTLRG (SEQ ID NO: 14); and/or

X₂ is selected from the group constituted by the sequences FL, FQ, FE, FP and YP; and/or

X₃ is selected from the group constituted by the sequences KYL, RYN, RFG, KYN, IEI and KEI; and/or

X₄ is selected from the group constituted by the sequences DRV, NNI, DNV, DTH, SKY; and/or

X₅ is selected from the group constituted by the sequences HKL, YKL, NKL, AVV and EYNVV (SEQ ID NO: 15); and/or

X₆ is selected from the group constituted by the sequences GDEFV (SEQ ID NO: 16), GSAA (SEQ ID NO: 17), GKRVP (SEQ ID NO: 18), GKIDH (SEQ ID NO: 19), NYTSRP (SEQ ID NO: 20) and GASLP (SEQ ID NO: 21); and/or

X₇ represents Y, I, S or A; and/or

X₈ is selected from the group constituted by the sequences IQYKSLKGL (SEQ ID NO: 22), SQYRSLKGM (SEQ ID NO: 23), VQYDAMNGL (SEQ ID NO: 24), IQYHSMEGL (SEQ ID NO: 25), VEAAKERDQ (SEQ ID NO: 26) and VQDARQLDH (SEQ ID NO: 27); and/or

X₉ is selected from the group constituted by the sequences PH, PY and AY; and/or

X₁₀ is selected from the group constituted by the sequences PTGKASVVLHNFLTSP (SEQ ID NO: 28), ANGQAAQVLHNFLSN (SEQ ID NO: 29), QEGHAAEELHQF (SEQ ID NO: 30), RTGSAAQMLHDFLSNP (SEQ ID NO: 31), YD and YDGGPEMLM (SEQ ID NO: 32).

In accordance with one preferred embodiment, the peptide is selected from the group constituted by a peptide comprising, or consisting of, a sequence of amino acids selected from the group constituted by the sequences

(SEQ ID NO: 1)
DFDPTEFKGPFPTIEICSKYCAVVCNYTSRPCYCVEAAKERDQWFPYCY
D,
(SEQ ID NO: 2)
GPFLTKYLCDRVCHKLCGDEFVCSCIQYKSLKGLWFPHCPTGKASVVLH
NFLTSP,
(SEQ ID NO: 3)
YFESPFETKYNCDTHCNKLCGKIDHCSCIQYHSMEGLWFPHCRTGSAAQ
MLHDFLSNP,
(SEQ ID NO: 4)
DIDPNTLRGPYPTKEICSKYCEYNVVCGASLPCICVQDARQLDHWFAYC
YDGGPEMLM,
(SEQ ID NO: 5)
SPFQTRYNCNNICHKLCGSAACACSQYRSLKGMWFPHCANGQAAQVLH
NFLSN,
and
(SEQ ID NO: 6)
YHYIESPFETRFGCDNVCYKLCGKRVPCSCVQYDAMNGLWFPHCQEGH
AAEELHQFL,

and functional variants thereof.

Preferably, the peptide is selected from the group constituted by a peptide comprising, or consisting of, a sequence of amino acids selected from the group constituted by the sequences SEQ ID NO: 1 to 4, and functional variants thereof. More particularly preferably, the peptide is selected from the group constituted by a peptide comprising, or consisting of, the sequence of amino acids SEQ ID NO: 1, and functional variants thereof.

The term “functional variant” refers to a peptide with formula (I) and for which the sequence differs from the parent protein by at least one substitution, insertion or deletion but which conserves a pesticidal activity, preferably an insecticidal activity.

Preferably, the functional variants have at least 50% sequence identity with the parent sequence, more particularly preferably at least 55%, 60%, 65%, 70%, 75%, 80% or 85% sequence identity with the parent sequence, and more particularly preferably at least 90%, 95%, 98% or 99% sequence identity with the parent sequence.

As used here, the term “sequence identity” or “identity” refers to the number (%) of pairings (identical amino acid residues) at positions originating from an alignment of two polypeptide sequences. The sequence identity is determined by comparing the sequences when they are aligned in a manner such as to maximize overlapping and identity while minimizing interruptions to the sequence. In particular, the sequence identity may be determined using any one of the many global or local alignment algorithms, depending on the length of the two sequences. Sequences with similar lengths are preferably aligned using global alignment algorithms (e.g. Needleman & Wunsch, J. Mol. Biol 48:443, 1970), which align the sequences in an optimal manner over the entire length, while sequences with substantially different lengths are preferably aligned using a local alignment algorithm, for example the algorithm from Smith and Waterman (Smith and Waterman, Adv. Appl. Math. 2:482, 1981) or the algorithm from Altschul (Altschul et al. (1997), Nucleic Acids Res. 25:3389-3402; Altschul et al. (2005) FEBS J. 272:5101-5109). Alignment with the aim of determining the percentage identity of the amino acid sequence may be carried out using any method that is known to the person skilled in the art, for example by using software that is available on websites such as http://blast.ncbi.nlm.nih.gov/or http://www.ebi.ac.uk/Tools/emboss/. The person skilled in the art will readily be able to determine the appropriate parameters for measuring the alignment. For the purposes of the present invention, the values for the percentage sequence identity of the amino acids designate values generated using the “Protein BLAST” (or Blastp) program in which the parameters are the default parameters (Expect threshold: 10, Word size: 6, Matrix=BLOSUM62, Gap Costs: Existence=11, Extension=1, Conditional compositional score matrix adjustment).

In accordance with certain embodiments, the functional variants may differ from the parent sequence by 1 to 10, i.e. 1, 2, 3, 4, 5, 6, 7, 8, 9 to 10 substitutions, insertions and/or deletions of amino acids, preferably by 1 to 5, i.e. 1, 2, 3, 4 or 5 substitutions, insertions and/or deletions of amino acids.

The term “substitution” as used here designates the replacement of one amino acid residue by another selected from the 20 standard natural amino acid residues, the rare natural amino acid residues and non-natural amino acids. Preferably, the term “substitution” refers to the replacement of one amino acid residue by another selected from the 20 standard natural amino acid residues (G, P, A, V, L, I, M, C, F, Y, W, H, K, R, Q, N, E, D, S and T). The substitution(s) may be conservative or non-conservative substitutions. The term “conservative substitution» as used in this document refers to a substitution of one residue of amino acid by another which has similar chemical or physical properties (size, charge or polarity). Examples of conservative substitutions include (i) basic amino acids (arginine, lysine and histidine), (ii) acidic amino acids (glutamic acid and aspartic acid), (iii) polar amino acids (glutamine and asparagine or serine, threonine and tyrosine), (iv) hydrophobic amino acids (methionine, leucine, isoleucine and valine), (v) aromatic amino acids (phenylalanine, tryptophan) and (vi) small amino acids (glycine, alanine). Preferably, the substitutions are conservative substitutions.

In accordance with one particular embodiment, the peptide comprises, or consists of, a sequence of amino acids selected from the group constituted by the sequences SEQ ID NO: 1 to 6, preferably comprises, or consists of, a sequence of amino acids selected from the group constituted by the sequences SEQ ID NO: 1 to 4, and more particularly preferably comprises, or consists of, the sequence of amino acids SEQ ID NO: 1.

The peptide used in accordance with the invention preferably has a size in the range 40 to 100 amino acids, more particularly preferably in the range 40 to 80 amino acids, and yet more particularly preferably in the range 40 to 70 amino acids. In accordance with one particular embodiment, the peptide used in accordance with the invention has a size of 50 to 60 amino acids.

The peptide used in accordance with the invention may comprise the sequence with formula (I) fused to another protein domain. This protein domain may, for example, be a signal sequence or a tag.

In particular, the peptide may be a precursor that undergoes post-translational modifications in order to obtain the nature and active form of the peptide. It may therefore comprise a translocation signal sequence and recognition sites and/or cleavage sites which enables it to undergo these post-translational modifications.

In accordance with one particular embodiment, the peptide is a precursor of a mature pesticide or insecticidal peptide, said precursor comprising a signal sequence for N-terminal translocation. The signal sequence may in particular be the sequence M-[R/K]-L-L-[Y/H]-G-F-L-I-I-M-L-T-[M/I]-[Y/H]-LS-[V/I]-Q (SEQ ID NO: 33). The peptide may also comprise a N-terminal or C-terminal tag that is useful for the purification or immobilization of the peptide. Tags of this type are well known to the person skilled in the art and include, for example, the tags histidine (His₆), FLAG, HA (epitope derived from the hemagglutinin of the influenza virus), MYC (epitope derived from the human proto-oncoprotein MYC) or GST (glutathione-S-transferase). Optionally, the peptide may comprise a site for cleavage by a protease or a chemical agent enabling this tag to be removed.

The peptide used in accordance with the invention may have one or more post-translational modifications and/or chemical modifications, in particular a glycosylation, an amidation, an acylation, an acetylation and/or a methylation.

In order to increase the resistance of the peptide to peptidases, protective groups may be added to the C-terminal and/or N-terminal ends. As an example, the protective group at the N-terminal end may be an acylation or an acetylation and the protective group at the C-terminal end may be an amidation or an esterification. The action of the proteases may also be counteracted, for example, by using amino acids with a D configuration or pseudo-peptide linkages replacing the “conventional” CONH peptide linkages, such as CHOH—CH₂, NHCO, CH₂—O, CH₂CH₂, CO—CH₂, N—N, CH═CH, CH₂NH, and CH₂—S.

The invention also encompasses the use of salts of said peptide, preferably salts which are acceptable in terms of plant protection. A salt that is acceptable in terms of plant protection is a salt that has no notable toxicity in the dose in which it is used, having regard to the plant, the environment or human beings. As an example, the salts could be salts with acceptable mineral acids such as hydrochloric acid, hydrobromic acid, sulfuric acid and phosphoric acid; salts with acceptable organic acids such as acetic acid, citric acid, maleic acid, malic acid, succinic acid, ascorbic acid or tartaric acid; salts with acceptable mineral bases such as sodium, potassium, calcium, magnesium or ammonium salts; or salts with organic bases that have a salifiable nitrogen. In accordance with certain preferred embodiments, the salt is an ammonium sulfate. These salts are routinely used and their preparation methods are well known to the person skilled in the art.

The invention concerns the use of the peptide as described above as a pesticidal agent, and more particularly as an insecticidal agent, preferably for plant protection applications.

As used here, the term “pesticide” refers to a compound intended to combat organisms that are considered to be harmful. When these organisms are insects, the term “insecticide” is used.

As used here, the term “plant protection application” refers to an action intended to protect plants and cultivated products against harmful organisms, in particular insects, and/or to ensure the conservation of cultivated products. The term “cultivated products” means products which are gathered or harvested, in particular vegetables, fruit, flowers or grain, and the transformed products that are derived from them, in particular flour.

Preferably, the peptide is used as an insecticidal agent in a plant protection treatment.

The insecticidal activity of the peptide can easily be tested by the person skilled in the art, in particular by observing the survival of insect larvae, for example pea aphid larvae, on a liquid medium containing the peptide to be tested. This routine technique is illustrated in the experimental section of the present application. Preferably, a peptide is considered to have an insecticidal activity when it has a LT₅₀ (lethal time at which mortality of individuals is 50%) of less than 5 days on insect larvae, preferably pea aphid larvae, at a concentration of 100 μM, preferably a concentration of 50 μM.

The insecticidal activity of the peptide with formula (I) is preferably against insect pests selected from the group constituted by hemiptera, lepidoptera, coleoptera and diptera.

Examples of hemiptera include, without being limited thereto, species of aphids such as Acyrthosiphon pisum, Aphis gossypii, Aulacorthum solani, Macrosiphum euphorbiae, Myzus persicae and Rhopalosiphum padi, cochineal insect species such as Planococcus citri, whitefly species such as Trialeurodes vaporariorum and Bemisia tabaci, or bug species such as Lygocoris pabulinus, Liocoris tripustulatus, Lygus rugulipennis and Corythucha ciliata.

Examples of lepidoptera include, without being limited thereto, caterpillars of several butterfly species such as Chrysodeixis chalcites, Mamestra brassicae, Spodoptera exigua, Clepsis spectrana, Cacoecimorpha prombana, Duponchelia fovealis, Tuta absoluta, Cydia pomonella, Lobesia botrana and Eupoecillia ambiguella.

Examples of coleoptera include, without being limited thereto, weevil species such as Sitophilus spp. And wireworm species such as Agriotes spp.

Examples of diptera include, without being limited thereto, species of flies such as Sciaridae spp., Musca domestica, Stomoxys calcitrans and Liriomyza spp.

In accordance with certain preferred embodiments, the peptide is used as an insecticidal agent against one or more hemiptera, and more particularly one or more aphids, in particular Acyrthosiphon pisum.

The peptide described above may be used alone or in combination with one or more other active agents. The additional active agent or agents may in particular be selected from the other peptides described above and with formula (I) and other pesticidal, insecticidal or plant protection agents, preferably with one or more other insecticidal agents.

Preferably, the peptide is used in combination with one or more other insecticidal peptides. In accordance with one particular embodiment, the peptide is used in combination with the insecticidal peptide AG41 (WO 2015/087238, Table II).

The present invention also concerns an expression cassette comprising a nucleic acid coding for a peptide as described above and with formula (I), operationally linked to a heterologous transcriptional promoter.

The term “expression cassette” designates a nucleic acid construct comprising a coding region and a regulatory region which are operationally linked. The expression “operationally linked” indicates that the elements are combined in a manner such that the expression of the coding sequence is under the control of the transcriptional promoter. Typically, the sequence for the promoter is placed upstream of the gene of interest, at a distance therefrom which is compatible with control of the expression.

The cassette may furthermore comprise other regulatory elements such as a transcription terminator, the sequence for a transit peptide and/or an activating sequence termed an “enhancer”.

As used here, the term “heterologous transcriptional promoter” refers to a promoter which, because of its nature, is not associated with a nucleic acid coding for the peptide. The cassette in accordance with the invention is thus a recombinant cassette.

The transcriptional promoter can readily be selected by the person skilled in the art as a function of the nature of the host cell in which expression is envisaged. In particular, this promoter may be a prokaryotic or eukaryotic, systemic or tissue-specific, inducible or constitutive promoter.

Preferably, the transcriptional promoter is a promoter allowing the expression of the peptide in a host plant cell or an entomopathogenic microorganism.

Alternatively, the cassette is intended to be inserted into a viral vector and the transcriptional promoter is a promoter enabling the expression of the peptide in a host cell infected by said vector, preferably a cell of an insect pest.

The expression cassettes in accordance with the invention may be constructed using conventional molecular biological techniques which are well known to the person skilled in the art.

The present invention also concerns an expression vector comprising an expression cassette in accordance with the invention. This expression vector may be used to transform a host cell and enable expression of the nucleic acid coding for a peptide as described above and with formula (I) in said cell.

The vector may be a DNA or an RNA, which may or may not be circular, or it may be single-stranded or double-stranded. It is advantageously selected from a plasmid, a phage, a phagemid, a virus, a cosmid and an artificial chromosome.

The person skilled in the art will readily be able to select the most appropriate vector, in particular having regard to the target host cell, the desired level of expression and the desired stability, the mode selected for introducing the vector into the host cell, etc.

The vector may also comprise elements enabling it to be selected in the host cell such as, for example, a gene for resistance to an antibiotic or to a herbicide, or a selection gene ensuring complementation of the respective gene that has been deleted in the genome of the host cell. Elements of this type are well known to the person skilled in the art and have been widely described in the literature.

The vectors in accordance with the invention may furthermore comprise an origin of replication and/or a sequence allowing targeted insertion into the genome of the host cell.

The vectors in accordance with the invention may be constructed using conventional molecular biological techniques which are well known to the person skilled in the art.

In accordance with a particular embodiment, the vector is a viral vector, preferably an entomopathogenic virus. Thus, the present invention concerns an entomopathogenic virus comprising a nucleic acid coding for a peptide as described above and with formula (I), said nucleic acid being placed under the control of a transcriptional promoter enabling expression of said nucleic acid in the host cell infected by the virus, the host cell preferably being an insect cell,

The entomopathogenic virus may be selected from the Baculoviridae, the Reoviridae, the Poxviridae, the Iridoviridae, the Parvoviridae, the Picornoviridae, and the Rhabdoviridae.

Preferably, the entomopathogenic virus belongs to the Baculoviridae family.

The transcriptional promoter can readily be selected by the person skilled in the art as a function of the target cell.

The present invention also concerns the use of an expression cassette or an expression vector in accordance with the invention in order to transform or transfect a cell. The host cell may be transformed/transfected in a transitory or stable manner and the cassette or the vector may be contained in the cell in the form of an episome or in the chromosomal form.

The present invention concerns a non-human host cell comprising a cassette or an expression vector in accordance with the invention.

It also concerns a host cell comprising a heterologous nucleic acid (which is not naturally present in the cell) coding for a peptide as described above and with formula (I). Said nucleic acid is preferably placed under the control of a transcriptional promoter enabling the expression of said nucleic acid in the cell. This promoter may be an endogenous promoter (naturally present in the cell) or a heterologous promoter (which is not naturally present in the cell).

The host cell may in particular be selected in order to act as a propagation vector for the peptide with formula (I) or for the purposes of production of the peptide.

The host cell may be a prokaryotic or eukaryotic cell. The host cell may in particular be a plant cell, an animal cell, a fungal cell or a bacterium.

In accordance with one embodiment, the host cell is an animal cell. In particular, the animal cell could be selected from the cells of mammals such as COS or CHO cells, the cells of insects such as Sf9, Sf21 or Hi5 cells, or the cells of protozoa or nematodes.

The cells of protozoa and of nematodes are preferably selected from entomopathogenic families, in particular the families used in biological control such as, for example; the families of the Amoebidae and Nosematidae protozoa or the nematode species Steinermatidae and Heterorhabditidae.

In accordance with another embodiment, the host cell is a cell of a microorganism, preferably a cell of an entomopathogenic microorganism and more particularly preferably an entomopathogenic bacterial cell or fungal cell.

Many bacteria have been described as being entomopathogenic. In particular, the bacteria may be selected from bacteria from the Bacillaceae, Enterobacteriaceae and Pseudomonaceae families. Preferably, the bacterium is selected from Bacillus thurengiensis and Bacillus sphaericus.

Similarly, many fungi have been described as being entomopathogenic. In particular, the fungus may be selected from fungi from the genuses Beauveria, for example Beauvaria bassiana, Metharizium, for example Metharizium anisopliae, Verticillium, Erynia, Hirsutella, Entomophtora and Entomophaga.

In accordance with yet another embodiment, the host cell is a plant cell, preferably a leguminous plant or cereal plant cell.

The plant cell may be a leguminous plant cell selected from the group constituted by soya, bean, pea, chickpea, peanut, lentil, alfalfa, feva bean and carob.

The plant cell may also be a cereal plant cell selected from the group constituted by wheat and corn.

The plant cell may also be a Nicotiana, for example Nicotiana sylvestris or Nicotiana tabacum.

The techniques for introducing a heterologous nucleic acid into these various types of cells (plant cell, animal cell, fungal cell or bacterial cell) are well known to the person skilled in the art.

In accordance with another aspect, the present invention also concerns a multicellular transgenic organism comprising a host cell in accordance with the invention, i.e. a cell comprising a heterologous nucleic acid coding for a peptide as described above and with formula (I), in particular comprising a cassette or an expression vector in accordance with the invention.

In accordance with one embodiment, the organism is a protozoon or a nematode. In accordance with one preferred embodiment, the organism is a transgenic plant or a multicellular plant structure As used here, the term “multicellular plant structure” refers to portions of a plant such as the flowers, seeds, leaves, stems, fruit, pollen, tubers, wood or multicellular structures such as the calluses, organs of the plant or immature embryos (for example explants used for transgenesis).

The reconstitution of a transgenic plant or of a multicellular plant structure as defined above from a transfected or transformed plant cell falls is within the scope of routine techniques which are well known to the person skilled in the art.

The peptide may be expressed ubiquitously (in all of the tissues of the plant) or only in certain tissues or organs, in particular the tissues or organs which are the targets of the pest organism.

All of the embodiments concerning the other aspects of the invention are also envisaged in this aspect.

The present invention also concerns a method for the production of a transgenic plant or of a multicellular plant structure comprising a host cell in accordance with the invention, said method comprising the introduction, into a plant cell, of a nucleic acid coding for a peptide as described above and with formula (I), of an expression cassette or of a vector in accordance with the invention, and the reconstitution of said plant or structure from said cell.

Optionally, the method may also comprise a step for selecting plants or structures containing the nucleic acid, the cassette or the vector. This selection may be carried out using any method known to the person skilled in the art, in particular by DNA amplification methods.

All of the embodiments concerning the other aspects of the invention are also envisaged in this aspect.

The present invention also concerns a method for the production of a peptide as described above and with formula (I), comprising the transformation or transfection of a cell by a nucleic acid coding for said peptide or a cassette or expression vector in accordance with the invention; culturing the transfected/transformed cell; and harvesting the peptide produced by said cell. Methods for the production of recombinant peptides are well known to the person skilled in the art.

The cell used for the production of the peptide may be a prokaryotic or a eukaryotic cell.

In accordance with one particular embodiment, the cell is a bacterium, in particular E. coli. Preferably, the bacterium used is capable of carrying out oxidative folding of the peptide, such as, for example, a strain of SHuffle E. coli (Lobstein et al. Microbial Cell Factories. 2012; 11:56).

The present invention also concerns a method for the production of a peptide as described above and with formula (I), comprising obtaining a transgenic plant in accordance with the invention and expressing the peptide, culturing this plant and harvesting the peptide produced by said plant.

Preferably, the transgenic plant is a Nicotiana, for example Nicotiana sylvestris or Nicotiana tabacum.

Methods for the production of recombinant peptides from transgenic plants are well known to the person skilled in the art.

The present invention also concerns a method for the production of a peptide as described above and with formula (I), comprising insertion of a nucleic acid coding for said peptide, of an expression cassette or an expression vector in accordance with the invention in an in vitro expression system, also termed acellular, and harvesting the peptide produced by said system. Many systems for in vitro or acellular expression are available commercially and the use of these systems is well known to the person skilled in the art.

The present invention also concerns a plant protection composition comprising at least one peptide as described above and with formula (I), a host cell or a transgenic organism in accordance with the invention and/or a virus in accordance with the invention, and an excipient which is acceptable in terms of plant protection. The invention furthermore concerns the use of a peptide as described above and with formula (I), a host cell or a transgenic organism in accordance with the invention and/or a virus in accordance with the invention, for the preparation of a plant protection composition.

The plant protection composition may be of any type. It may be an aqueous or hydro-alcoholic solution, a paste, a gel, in particular an aqueous gel (or hydrogel), a glue, a foam, a water-in-oil emulsion or an oil-in-water emulsion, a multiple emulsion, a micro-emulsion or nano-emulsion, a micellar solution, a suspension, or a colloid. The plant protection composition may also be solid, for example in the form of a powder or granules which can be applied directly by “dusting” or which have to be dissolved or dispersed in a suitable solvent before application.

The term “excipient which is acceptable in terms of plant protection” means an excipient that does not have any notable toxicity, in the dose in which it is used, as regards the plant, the environment and human beings. Excipients that can be used in the plant protection compositions are well known to the person skilled in the art and include, inter alia, diluents and fillers, wetting agents, surfactants, for example ionic, amphoteric or non-ionic surfactants, dispersing agents, thickening agents, gelling agents, agents allowing the controlled release of the active agents, for example encapsulation agents or micellar agents such as phospholipids, thixotropic adjuvants, colorants, antioxidants, preservatives, stabilizers, film-forming agents, vehicles, in particular solvents such as water and lower alcohols, oils of animal, vegetable or mineral origin, resins, waxes, rosin, latex, gums such as gum Arabic, anti-foaming agents, and adhesive agents.

The plant protection composition in accordance with the invention may furthermore comprise one or more additional plant protection agents. The additional plant protection agent or agents may, for example, be selected from insecticides, bactericides, fungals, virucides, growth regulators or stimulators of the natural defenses of the plant such as elicitors.

The galenical form and the excipients of the plant protection composition in accordance with the present invention can readily be selected by the person skilled in the art and essentially depend on the infestation to be treated and/or the mode of administration.

The present invention also concerns the use of a peptide as described above and with formula (I) or of a plant protection composition in accordance with the invention, for the treatment or prevention of infestation of a plant and/or a cultivated product by pest organisms, preferably by insects, the method comprising bringing the peptide as described above and with formula (I) into contact with said plant, cultivated product or pest organism.

All of the references cited in this description are incorporated into the present application by reference. Other characteristics and advantages of the invention will become apparent from the examples below, which are given by way of non-limiting illustration.

### Examples

### Method and Apparatus

### Synthesis and Purification of BCR4 Peptide

Ten milligrams of non-functional reduced unrefined BCR4 peptide (DFDPTEFKGPFPTIEICSKYCAVVCNYTSRPCYCVEAAKERDQWFPYCYD; SEQ ID NO: 1) was obtained from Proteogenix (Strasbourg, France). Oxidative folding of the peptide in vitro was carried out using the protocol described above (cf. Da Silva and al, 2009). In brief, the oxidative folding was carried out by incubating the reduced peptide (10 mg) in a deoxygenated buffer containing 0.1 mM of oxidized glutathione (10 equiv.), 1 mM de glutathione (100 equiv.), 1 mM of EDTA, 100 mM TRIS, pH 8.5, at 20° C., for 48 h in an atmosphere of argon (final concentration of peptide 30 μM). The reaction was acidified by adding TFA (200 μL) and the unrefined mixture was purified by semi-preparative HPLC in order to produce pure BCR4 (2.2 mg, i.e. 22% yield). The purity of the BCR4 peptide was evaluated by RP-HPLC, its mass was obtained by high resolution mass spectrometry (ESI-HRMS). The concentration of peptide was determined by measuring the surface area of its RP-HPLC peak at 214 nm.

### Insecticide Tests

The experiments were carried out on aphids from the parthenogenetic clone of the pea aphid LL01 which contained only the primary symbiont B. aphidicola. The aphids were bred and kept on feva bean plants (Vicia faba L., Aquadulce variety) under climate-controlled conditions (temperature 21±1° C., relative humidity 70%, photoperiod of 16 h), in order to obtain aphids which reproduced solely by viviparous parthenogenesis. In order to reduce the variability linked to the use of individuals at different stages of development, the aphids were obtained from a synchronized brood. For this, adult aphids that were ready to lay were placed on feva bean plants. After 24 hours, the young larvae were recovered and placed on an artificial medium which contained or did not contain the peptide of interest. The manipulation was carried out with a negative control composed solely of artificial medium and a positive control representing one of the concentrations of the range being studied with the aphids. The range being studied comprised different media containing the BCR4 peptide studied at various concentrations which varied from 5 to 80 μM. The experiments were carried out on thirty larvae per condition divided into three distinct groups of ten individuals.

### Statistical Analyses

All of the data regarding the mortality of the aphids were analyzed statistically by standard survival processing. All of the BCR4 concentrations were analyzed separately in a parametric survival analysis with a normal logarithmic adjustment.

### Analyses of Sequences

The orthologous sequences of the BCR proteins were recovered using a combination of TBLASTN and BLASTP (Johnson and al, 2008) against the genomes of aphids available in the AphidBase database (Legeai et al., 2010) and the entire non-redundant proteins, nucleotides and EST database from NCBI. The BCR proteins underwent multiple sequence alignments using the MUSCLE program (Edgar et al., 2004).

### Results

### Synthesis and Purification of BCR4 Peptide

2.2 mg of pure BCR4 peptide was obtained by a chemical synthesis in the solid phase followed by oxidative folding in order to form the C1-C5, C2-C4 and C3-C6 disulfide bridges (FIG. 1).

The purity of the BCR4 peptide was evaluated using RP-HPLC and its mass was obtained by high resolution mass spectrometry (ESI-HRMS): the theoretical mass calculated for BCR4 was 5891.5400 g·mol⁻¹; the experimental mass obtained was 5891.5437 g·mol⁻¹ (FIG. 2A). The concentration of peptide was determined by measuring the surface area of its RP-HPLC peak at 214 nm (FIG. 2B).

### Insecticide Tests

The measurement of the insecticidal activity of BCR4 was carried out by incorporating increasing doses of peptides into the nutrient medium for the aphids and monitoring of their mortality was carried out over 7 days. A very significant dose-response over the survival time for the aphids was observed (TLSO: lethal time at which mortality of individuals is 50%) (Table 1).

TABLE 1
Toxicity of BCR4 peptide on the pea aphid Acyrthosiphon pisum.
TL50 in days, with confidence intervals, obtained by survival
analysis with a normal logarithmic adjustment.

| BCR4 | Concentration (μM) | | | | | | |
|---|---|---|---|---|---|---|---|
| peptide | 80 | 40 | 35 | 27.5 | 20 | 10 | 5 |
| TL50 | 1.16 | 1.76 | 1.94 | 3.24 | 3.48 | 11.3 | >20 |
| (days) | [0.98- | [1.27- | [1.47- | [2.22- | [2.28- | [4.16- | |
| | 1.37] | 2.42] | 2.57] | 4.72] | 5.31] | 30.8] | |

The dose-response effect commenced very early, from the first day following ingestion, with a high mortality. This range of activity (5-80 μM) was similar to that of the peptide AG41, an entomotoxic promoter of plant origin (cf. WO 2015/087238).

### Sequence Analysis

By means of an exhaustive search of the nucleotide and protein databases from the NCBI coupled with a specific inspection of sequences for aphid species available in the AphidBase (Legeai et al., 2010), the inventors recovered a total of 32 BCR sequences. All of these sequences were identified in aphid species. The 32 BCR species were used to align the amino acid sequences and, according to the consensus motifs obtained following these alignments, it appears that the BCR sequences can be grouped into four sub-families comprising the sequences (i) BCR1-2-4-5, (ii) BCR3, (iii) BCR6 and (iv) BCR8 (Table 2).

TABLE 2
Characteristics of four BCR sub-families.

| | Number of | | Orthologs identified in |
|---|---|---|---|
| | cysteine | | aphids for which genome |
| | residues | Consensus motif^{a} | has been sequenced^{b} |
| BCR1-2-4-5 | 6 | C(3)C(3-5)C(4-6)C(1)C(13)C | Apis (4), Dnox (1) |
| BCR3 | 6 | C(3)C(6)C(21-25)C(6)C(4)C | Agly (1), Apis (1), Dnox (2), |
| | | | Mcer (1), Mper (1), Rpad (1) |
| BCR6 | 8 | C(15)C(3)C(3)C(10)C(10)C(1)CC | Apis (1), Dnox (2), Mcer (1), |
| | | | Mper (1) |
| BCR8 | 6 | C(3-4)C(10)C(17)C(1)CC | Agly (1), Apis (1), Dnox (1), |
| | | | Mcer (1), Mper (1), Rpad (1) |

^{a}The number of residues between the cysteines is noted between parentheses.
^{b}Abbreviations: Agly, Aphis glycines; Apis, Acyrthosiphon pisum; Dnox, Diuraphis noxia; Mcer, Myzus cerasi; Mper, Myzus persicae; Rpad, Rhopalosiphum padi.

The peptides BCR1 (SEQ ID NO: 2), BCR2 (SEQ ID NO: 3), BCR4 (SEQ ID NO: 1) and BCRS (SEQ ID NO: 4) of Acyrthosiphon pisum, as well as two peptides from Acyrthosiphon kondoi (SEQ ID NO: 6) and from Diuraphis noxia (SEQ ID NO: 5), belong to the sub-family BCR1-2-4-5 which had a strictly conserved consensus motif (FIG. 3).

### BIBLIOGRAPHIC REFERENCES

Da Silva P et al., 2009. Biopolymers 92(5): 436-444.
Edgar R C et al., 2004. MUSCLE: multiple sequence alignment with high accuracy and high throughput. Nucleic Acids Res. 32, 1792-1797.
Johnson M et al., 2008. NCBI BLAST: a better web interface. NCBI BLAST: a better web interface. Nucleic Acids Res. 2008 Jul. 1; 36 (Web Server issue):W5-9. Legeai F et al., 2010. AphidBase: a centralized bioinformatic resource for annotation of the pea aphid genome. Insect Mol. Biol. 19, 5-12.
Shigenobu and Stern. Proc. R. Soc. B. 2013 280, 20121952.

1-17. (canceled)

18. A method of protecting a plant against an insect comprising applying, to the plantas an insecticidal agent, a peptide selected from the group consisting of:
a peptide comprising a sequence of amino acids selected from the group consisting of SEQ ID NO: 1, 2, 3, 4, 5 and 6, and
a peptide comprising a sequence with formula (I), having at least 80% sequence identity with one of SEQ ID NO: 1, 2, 3, 4, 5 or 6, and having an insecticidal activity, said sequence with formula (I) being [chemical structure]
in which
X₁ represents a sequence of 1 to 9 amino acids,
X₂ represents a sequence of 2 amino acids,
X₃ represents a sequence of 3 amino acids,
X₄ represents a sequence of 3 amino acids,
X₅ represents a sequence of 3 to 5 amino acids,
X₆ represents a sequence of 4 and 6 amino acids,
X₇ represents one amino acid,
X₈ represents a sequence of 9 amino acids,
X₉ represents a sequence of 2 amino acids,
X₁₀ represents a sequence of 2 to 16 amino acids,
or a salt of said peptide which is acceptable in terms of plant protection.

19. The method of claim 18, wherein the peptide comprises SEQ ID NO: 1, 2, 3, 4, 5 or 6.

20. The method of claim 18, wherein the peptide comprises SEQ ID NO: 1, 2, 3 or 4.

21. The method of claim 20, wherein the peptide comprises SEQ ID NO: 1.

22. The method of claim 18, in which the size of the peptide is in the range 40 to 100 amino acids.

23. The method of claim 22, in which the size of the peptide is in the range 40 to 70 amino acids.

24. The method of claim 18, in which the peptide is used as an insecticidal agent against hemiptera, lepidoptera, coleoptera and/or diptera.

25. The method of claim 24, in which the peptide is used as an insecticidal agent against hemiptera.

26. The method of claim 18, in which the peptide is used as an insecticidal agent against aphids.

27. The method of claim 26, wherein the aphids are Acyrthosiphon pisum.

28. A heterologous nucleic acid comprising a nucleic acid coding for a peptide, said nucleic acid being placed under the control of a transcriptional promoter enabling the expression of said nucleic acid in a cell and said peptide being:
a sequence of amino acids selected from the group consisting of SEQ ID NO: 1, 2, 3, 4, 5 and 6, or
a sequence of formula (I), said sequence having at least 80% sequence identity with one of SEQ ID NO: 1, 2, 3, 4, 5 or 6 and having an insecticidal activity, said sequence with formula (I) being [chemical structure]
in which
X₁ represents a sequence of 1 to 9 amino acids,
X₂ represents a sequence of 2 amino acids,
X₃ represents a sequence of 3 amino acids,
X₄ represents a sequence of 3 amino acids,
X₅ represents a sequence of 3 to 5 amino acids,
X₆ represents a sequence of 4 and 6 amino acids,
X₇ represents one amino acid,
X₈ represents a sequence of 9 amino acids,
X₉ represents a sequence of 2 amino acids,
X₁₀ represents a sequence of 2 to 16 amino acids,

29. A method for the production of a transgenic plant, comprising introducing a heterologous nucleic acid according to claim 28 into a plant cell, and reconstituting said plant from said cell.

30. An entomopathogenic virus comprising a heterologous nucleic acid of claim 28, said nucleic acid being placed under the control of a transcriptional promoter enabling the expression of said nucleic acid in the insect infected by the virus.

31. A non-human host cell comprising a heterologous nucleic acid of claim 28.

32. The host cell of claim 31, in which the cell is an entomopathogenic microorganism, an entomopathogenic bacterium or an entomopathogenic fungus.

33. The host cell of claim 31, in which the cell is:
a) a plant cell;
b) a leguminous plant or cereal plant cell;
c) a leguminous plant cell selected from the group consisting of soya, bean, pea, chickpea, peanut, lentil, alfalfa, feva bean and carob; or
d) a cereal plant cell selected from the group consisting of wheat and corn.

34. A transgenic plant or multicellular plant structure comprising at least one cell as claimed in claim 31.

35. A plant protection composition comprising at least one peptide, said at least one peptide comprising a sequence of amino acids selected from the group consisting of SEQ ID NO: 1, 2, 3, 4, 5 and 6, or
a sequence with formula (I) having at least 80% sequence identity with one of SEQ ID NO: 1, 2, 3, 4, 5 or 6, and having an insecticidal activity, said sequence with formula (I) being [chemical structure]
in which
X₁ represents a sequence of 1 to 9 amino acids,
X₂ represents a sequence of 2 amino acids,
X₃ represents a sequence of 3 amino acids,
X₄ represents a sequence of 3 amino acids,
X₅ represents a sequence of 3 to 5 amino acids,
X₆ represents a sequence of 4 and 6 amino acids,
X₇ represents one amino acid,
X₈ represents a sequence of 9 amino acids,
X₉ represents a sequence of 2 amino acids,
X₁₀ represents a sequence of 2 to 16 amino acids, or one of its acceptable salts in terms of plant protection, a host cell expressing said at least one peptide, and/or a virus comprising a heterologous nucleic acid encoding said at least one peptide, and a support and/or excipient which is acceptable in terms of plant protection.

36. The plant protection composition of claim 35, further comprising one or more additional plant protection agents.

37. The plant protection composition of claim 36, wherein the additional plant protection agent is selected from insecticides, bactericides, fungals, virucides, growth regulators or stimulators of the natural defenses of the plant.


SEQUENCE LISTING (33 sequences)

SEQ ID NO: 1 - protein, 50 aa, Acyrthosiphon pisum
DFDPTEFKGPFPTIEICSKYCAVVCNYTSRPCYCVEAAKERDQWFPYCYD

SEQ ID NO: 2 - protein, 55 aa, Acyrthosiphon pisum
GPFLTKYLCDRVCHKLCGDEFVCSCIQYKSLKGLWFPHCPTGKASVVLHNFLTSP

SEQ ID NO: 3 - protein, 58 aa, Acyrthosiphon pisum
YFESPFETKYNCDTHCNKLCGKIDHCSCIQYHSMEGLWFPHCRTGSAAQMLHDFLSNP

SEQ ID NO: 4 - protein, 58 aa, Acyrthosiphon pisum
DIDPNTLRGPYPTKEICSKYCEYNVVCGASLPCICVQDARQLDHWFAYCYDGGPEMLM

SEQ ID NO: 5 - protein, 53 aa, Diuraphis noxia
SPFQTRYNCNNICHKLCGSAACACSQYRSLKGMWFPHCANGQAAQVLHNFLSN

SEQ ID NO: 6 - protein, 57 aa, Acyrthosiphon kondoi
YHYIESPFETRFGCDNVCYKLCGKRVPCSCVQYDAMNGLWFPHCQEGHAAEELHQFL

SEQ ID NO: 7 - protein, 9 aa, Artificial Sequence
  amino acid sequence represented by “X8”
  misc_feature (1)..(1): Xaa can be any naturally occurring amino acid
  MISC_FEATURE (2)..(2): X is E or Q
  misc_feature (3)..(7): Xaa can be any naturally occurring amino acid
  MISC_FEATURE (8)..(8): X is D or G
  misc_feature (9)..(9): Xaa can be any naturally occurring amino acid
XXXXXXXXX

SEQ ID NO: 8 - protein, 9 aa, Artificial Sequence
  amino acid sequence represented by “X8”
  misc_feature (1)..(1): Xaa can be any naturally occurring amino acid
  misc_feature (3)..(7): Xaa can be any naturally occurring amino acid
  misc_feature (9)..(9): Xaa can be any naturally occurring amino acid
XEXXXXXDX

SEQ ID NO: 9 - protein, 9 aa, Artificial Sequence
  amino acid sequence represented by “X8”
  MISC_FEATURE (1)..(1): x is V, I or S
  MISC_FEATURE (2)..(2): x is E or Q
  MISC_FEATURE (3)..(3): x is A, Y or D
  MISC_FEATURE (4)..(4): x is A, H, D, R or K
  MISC_FEATURE (5)..(5): x is K, S, A or R
  MISC_FEATURE (6)..(6): x is E, M, Q or L
  MISC_FEATURE (7)..(7): x is R, E, L, N or K
  MISC_FEATURE (8)..(8): x is D or G
  MISC_FEATURE (9)..(9): x is Q, L, H or M
XXXXXXXXX

SEQ ID NO: 10 - protein, 9 aa, Artificial Sequence
  amino acid sequence represented by “X8”
VEAAKERDQ

SEQ ID NO: 11 - protein, 6 aa, Artificial Sequence
  amino acid sequence represented by “X1”
YHYIES

SEQ ID NO: 12 - protein, 4 aa, Artificial Sequence
  amino acid sequence represented by “X1”
YFES

SEQ ID NO: 13 - protein, 9 aa, Artificial Sequence
  amino acid sequence represented by “X1”
DFDPTEFKG

SEQ ID NO: 14 - protein, 9 aa, Artificial Sequence
  amino acid sequence represented by “X1”
DIDPNTLRG

SEQ ID NO: 15 - protein, 5 aa, Artificial Sequence
  amino acid sequence represented by “X5”
EYNVV

SEQ ID NO: 16 - protein, 5 aa, Artificial Sequence
  amino acid sequence represented by “X6”
GDEFV

SEQ ID NO: 17 - protein, 4 aa, Artificial Sequence
  amino acid sequence represented by “X6”
GSAA

SEQ ID NO: 18 - protein, 5 aa, Artificial Sequence
  amino acid sequence represented by “X6”
GKRVP

SEQ ID NO: 19 - protein, 5 aa, Artificial Sequence
  amino acid sequence represented by “X6”
GKIDH

SEQ ID NO: 20 - protein, 6 aa, Artificial Sequence
  amino acid sequence represented by “X6”
NYTSRP

SEQ ID NO: 21 - protein, 5 aa, Artificial Sequence
  amino acid sequence represented by “X6”
GASLP

SEQ ID NO: 22 - protein, 9 aa, Artificial Sequence
  amino acid sequence represented by “X8”
IQYKSLKGL

SEQ ID NO: 23 - protein, 9 aa, Artificial Sequence
  amino acid sequence represented by “X8”
SQYRSLKGM

SEQ ID NO: 24 - protein, 9 aa, Artificial Sequence
  amino acid sequence represented by “X8”
VQYDAMNGL

SEQ ID NO: 25 - protein, 9 aa, Artificial Sequence
  amino acid sequence represented by “X8”
IQYHSMEGL

SEQ ID NO: 26 - protein, 9 aa, Artificial Sequence
  amino acid sequence represented by “X8”
VEAAKERDQ

SEQ ID NO: 27 - protein, 9 aa, Artificial Sequence
  amino acid sequence represented by “X8”
VQDARQLDH

SEQ ID NO: 28 - protein, 16 aa, Artificial Sequence
  amino acid sequence represented by “X10”
PTGKASVVLHNFLTSP

SEQ ID NO: 29 - protein, 15 aa, Artificial Sequence
  amino acid sequence represented by “X10”
ANGQAAQVLHNFLSN

SEQ ID NO: 30 - protein, 12 aa, Artificial Sequence
  amino acid sequence represented by “X10”
QEGHAAEELHQF

SEQ ID NO: 31 - protein, 16 aa, Artificial Sequence
  amino acid sequence represented by “X10”
RTGSAAQMLHDFLSNP

SEQ ID NO: 32 - protein, 9 aa, Artificial Sequence
  amino acid sequence represented by “X10”
YDGGPEMLM

SEQ ID NO: 33 - protein, 19 aa, Artificial Sequence
  Signal sequence
  MISC_FEATURE (2)..(2): x is R or K
  MISC_FEATURE (5)..(5): x is Y or H
  MISC_FEATURE (14)..(14): x is M or I
  MISC_FEATURE (15)..(15): x is Y or H
  MISC_FEATURE (18)..(18): x is V or I
MXLLXGFLIIMLTXXLSXQ